(19) United States
(12) Patent Application Publication
(10) Pub. No.: US 2016/0008165 A1
(43) Pub. Date: Jan. 14, 2016

(54) TRANSPARENT WARMING COVER FOR SHORT TERM TEMPERATURE REGULATION OF MEDICAL PATIENTS

(71) Applicants: Camille BAELDEN, Cambridge,, MA (US); Teja INAMDAR, Cambridge,, MA (US); David MCCALIB, Hillsboro,, OR (US); Steven OBIAJULU, Westfield,, NJ (US); Mathieu PICARD, Cambridge,, MA (US); Michael M. FUENFER, (US)

(72) Inventors: Camille Baelden, Cambridge, MA (US); Tejas Inamdar, Cambridge, MA (US); David McCalib, Hillsboro, OR (US); Steven Obiajulu, Westfield, NJ (US); Mathieu Picard, Cambridge, MA (US); Michael M. Fuenfer, Boston, MA (US)

(73) Assignee: THE GENERAL HOSPITAL CORPORATION, Boston, MA (US)

(21) Appl. No.: 14/773,079

(22) PCT Filed: Mar. 7, 2014
(86) PCT No.: PCT/US2014/021739

§ 371 Date: Sep. 4, 2015

Related U.S. Application Data
(60) Provisional application No. 61/774,181, filed on Mar. 7, 2013.

(51) Int. Cl. A61F 7/00; H05B 3/02; H05B 3/16; H05B 3/12; H05B 3/34; H05B 3/00; H05B 3/06

(52) U.S. Cl. CPC A61F 7/0097; A61F 7/007; H05B 3/0014; H05B 3/026; H05B 3/06; H05B 3/12; H05B 3/34; H05B 3/16; H05B 3/02; A61F 2007/0071; A61F 2007/0078

(57) ABSTRACT

A transparent portable warming device to insure normothermia during medical transport, particularly for transporting pediatric patients and infants is disclosed. The warming device includes a one or more layers of a thin, flexible, transparent material. A heating element includes a parallel circuit of resistive wires controlled by a battery-powered power controller. The controller maintains the temperature of the blanket at a predetermined temperature level for periods of time suitable for transporting patients. In one embodiment, the resistive wires are nichrome, and the flexible material comprises polyvinylchloride. The system can be powered by a lightweight twelve volt battery.

### CROSS-REFERENCE TO RELATED APPLICATIONS

This application claims the benefit of U.S. Provisional Patent Application Ser. No. 61/774,181 filed on Mar. 7, 2013, which is incorporated herein by reference in its entirety.

### BACKGROUND

Maintaining the body temperature of patients at a suitable level to prevent hypothermia and other conditions is important in a many areas of medical care, and is particularly in pediatric care. Infants and young children have diminished ability to conserve body heat as a result of a number of anatomic and physiologic factors including thin dermis, a paucity of subcutaneous fat, diminished body fat stores, immaturity of hypothalamic function and hormonal secretion, and a disproportionately large head to body surface area. Hypothermia may be associated with profound adverse pathophysiologic effects including coagulopathy, impaired enzymatic function, changes in cerebral blood flow, increased oxygen consumption and decreased oxygen transport to vital organs as well as patient discomfort.

There are numerous methods for maintaining normal core body temperature (normothermia) in pediatric patients in the controlled environment of an intensive care unit or operating room. Radiant heaters, thermostatically controlled patient rooms, warmed intravenous fluids, forced-air warmers, heated blankets, isolettes (neonates only) and a variety of other devices are in common use. However, pediatric and other patients are susceptible to hypothermia when transported from a temperature-controlled environment to other areas of a hospital such as imaging suites and operating rooms, or when transported from one medical facility to another by ground or air. During these periods in uncontrolled environments, there are limited means currently available for maintaining patients in a normothermic state.

Complicating the need to maintain body temperature during transport, patients are often connected to devices for infusion of medications and intravenous fluids, or have tubes inserted into the trachea, stomach, bladder and other body cavities. In addition, leads are routinely attached to the skin for monitoring of body temperature, heart rate and rhythm, respirations, oxygen saturation and blood pressure. Should any of these numerous connections become dislodged, the results could be catastrophic. Therefore, direct visualization of the patient's chest, abdomen and extremities is critical not only to ensure intact connections, but also to observe chest excursion, skin perfusion, and other aspects that ensure patient safety.

The present disclosure addresses a need for a portable warming device to prevent hypothermia through radiant and convective heat loss, and a need for a portable blanket that permits continuous observation of the thorax and extremities during transport, both for patient observation and to assure that tubes and monitoring leads do not become dislodged during transport.

### SUMMARY OF INVENTION

In one aspect of the invention, a transparent warming blanket is provided which includes at least one flexible transparent layer, a plurality of resistive wires integrated with the transparent layer, and a battery electrically connected to the resistive wires so as to provide sufficient power to induce heat from the resistive wires, wherein heat produced from the wires is sufficient to achieve and maintain a steady state temperature of about 40 degrees Celsius beneath a substantial portion of the blanket.

In another aspect of the invention, the warming blanket is configured to provide a steady state temperature of about 40 degrees within an environment having an ambient temperature of about 23 degrees Celsius or less. In one embodiment, the steady state temperature can be achieved, for example, within a time period of about 14 minutes. In another embodiment, the steady state temperature can be achieved within a time period of about 2 minutes.

In an embodiment, the plurality of resistive wires comprises nichrome. The at least one flexible transparent layer can comprises a polyvinyl chloride (PVC). In another embodiment, the at least one flexible transparent layer can comprise two transparent layers. The resistive wires can be disposed between the layers.

The warming device can include a digital controller to regulate the power output of the resistive wires. In an embodiment, the blanket further includes a multichannel MOSFET electrically connected to the resistive wires and the controller, the controller and MOSFET configured to produce a duty cycle limiting the maximum power output to limit a surface temperature output to directly adjacent skin of about 37 degrees Celsius or less.

In one embodiment of the invention, the battery provides a power output of about 12 Volts. In another embodiment, the battery can provide a power output of about 24 Volts.

In an embodiment, the blanket is fully portable, such as for use in transporting a patient under the blanket to or within a medical facility while being warmed. The battery can, for example, weigh about 1.5 Kg or less.

In an embodiment, the at least one transparent layer is sufficiently sized to cover at least an infant. In another embodiment, the at least one transparent layer is sufficiently sized to cover at least an adult. To accommodate the size of the blanket, the battery may be appropriately powered and scaled to in order to warm it according to embodiments herein.

In an embodiment, the warming blanket can alternatively be powered from an indefinitely dedicated power source. For example, the blanket could be plugged into and powered from a traditional wall socket in order to initially or indefinitely warm the blanket, then unplugged and switched to battery power while the blanket and person thereunder is transported or moved.

These and other features and advantages of the present invention will become apparent upon reading the following detailed description when taken in conjunction with the drawings.

### BRIEF DESCRIPTION OF THE DRAWINGS

FIG. 1 is a block diagram of a warming device for temperature regulation of medical patients constructed in accordance with one embodiment of the disclosure.

FIG. 2 is a top view of a warming cover or blanket as shown in the block diagram of FIG. 1.

FIG. 3 is a top view of a portion of the warming cover or blanket of FIG. 2, illustrating a heating element.

FIG. 4 is a side view of a patient on a bed and covered with a warming blanket constructed in accordance with one embodiment of the present disclosure, and illustrating heat transfer from the blanket.

FIG. 5 is a resistive network model of the radiation and convention heat flows of FIG. 4.

FIG. 6 is a partial view of the blanket of FIG. 2, illustrating portions used in calculations for a one dimensional temperature distribution model of the blanket.

FIG. 7 is a chart illustrating wire spacing versus temperature increase for the heating wires in a blanket constructed in accordance with the present disclosure.

FIG. 8 is a chart illustrating heating time versus heating power for a warming blanket constructed in accordance with the present disclosure.

### DETAILED DESCRIPTION OF THE DISCLOSURE

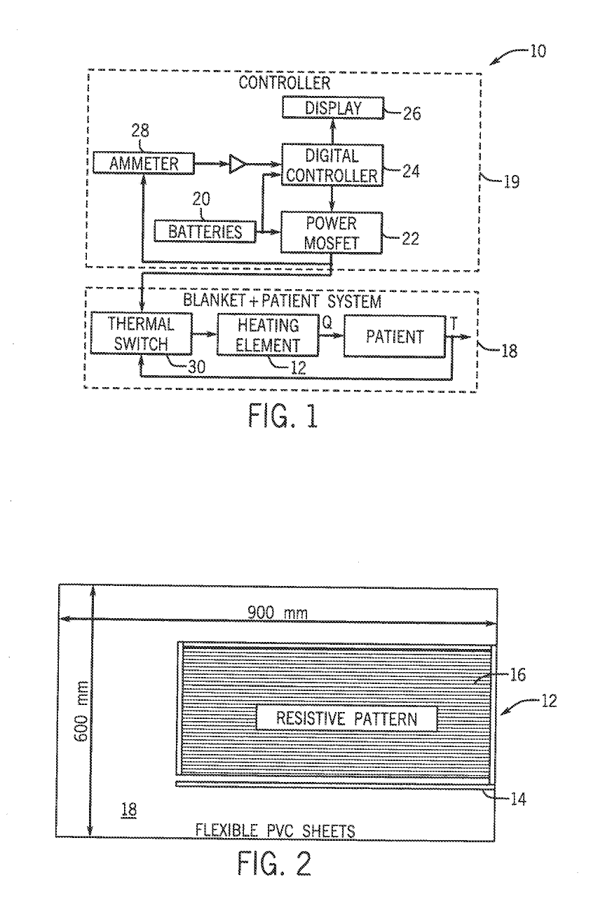

Referring now to the Figures and more particularly to FIGS. 1 and 2, a transparent portable warming device 10 to insure normothermia during medical transport is shown. The device 10 consists of a warming cover or blanket 18 which can be made from one or more layers of a thin, flexible, transparent material. A heating element 12 is coupled to the blanket 18, and is coupled to a power controller 19, which is preferably powered by batteries 20 to assure portability. The controller 19 maintains the heat of the blanket 18 at a predetermined temperature level, as described below. The controller 19 can be connected to the heating element 12 in the blanket 18 through a thermal switch 30, which can be used to limit the temperature of the blanket 18 to prevent overheating or burning.

Referring still to FIG. 1, in one embodiment, the power controller 19 includes a digital controller 24, which can be a microprocessor, microcontroller, or other suitable device. The digital controller 24 can control the power supplied to the heating element 12 in the blanket 18 by varying the duty cycle of pulse width modulation using an electronic switching device, such as a power MOSFET 22. Current supplied to the heating element 12 can be monitored through an ammeter 28 in communication with the digital controller 24. The digital controller 24 further monitors voltage across the battery 20 to check for blanket damage, disconnection, and low battery conditions. The digital controller 24 can also be programmed to control the exposure time using a timer. A display 26 can be connected to the digital controller 24 to allow for monitoring of the device. Other types of data monitoring equipment, such as wireless communications transmitting temperature data, can be used to monitor the temperature of the blanket 18 or other conditions.

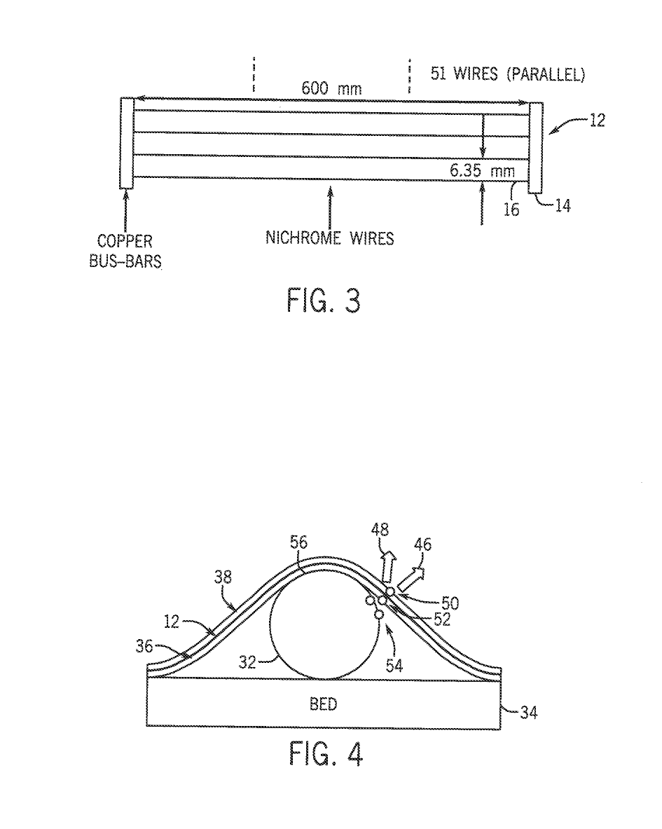

Referring again to FIG. 2 and now also to FIG. 3, the heating element 12 can be a circuit including a plurality of parallel resistive wires 16, which can be connected between peripheral bus bars 14, resulting in a circuit of parallel resistances. The heating element 12 preferably extends through a large portion of the blanket 18 to maintain temperature uniformity, but the wires 16 are spaced apart to allow clear visibility through the blanket 18.

Referring now to FIG. 4, a side view of one embodiment of the warming device 10 is shown in use. A patient 32 is shown on a bed 34, which can be a stationary bed, or a patient transport device. The blanket 18 comprises an outer layer 38 and an inner layer 36 of a flexible transparent material. The heating element 12 is positioned between the layers, and is controller by a power controller 19 as described above.

Referring still to FIG. 4, a heat transfer analysis can be used to calculate the heating power required to achieve a selected heating temperature, and to evaluate the temperature of the blanket 18 on the skin of the patient 32. This analysis helps to minimize or eliminate the potential for overheating or burns. The power required to be applied by the controller 19 for the blanket 18 can be calculated to maintain normal body temperature in steady state, and also to determine an initial heating time and applied power level to initially reach the desired temperature of the blanket 18. Because the temperature of the heating wire 16 should not exceed a pre-determined safe temperature for the selected patient, a heat distribution model can also be advantageously used to calculate the temperature distribution of the blanket 18 as a function of spacing of the wires 16. The temperature distribution can be modeled, for example, for the areas of the blanket directly in contact with the skin of a patient using the warming device 10.

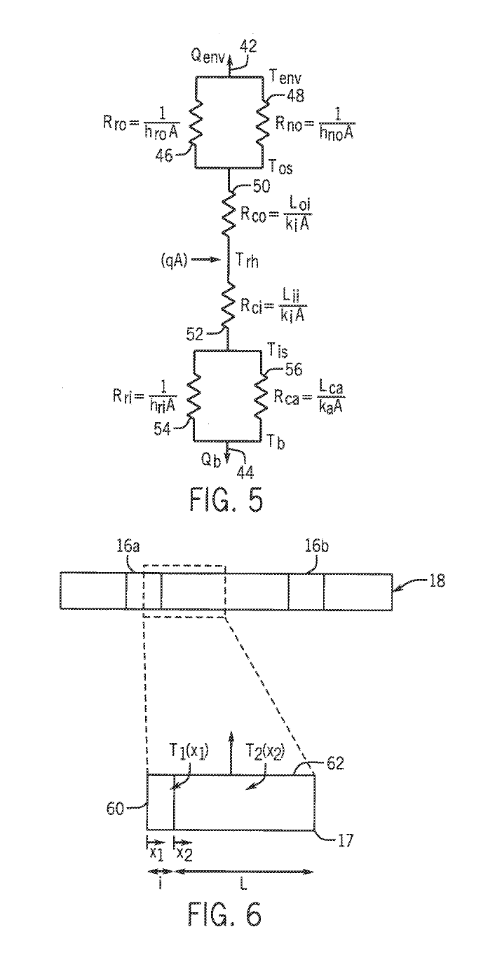

Referring still to FIG. 4, and also to FIG. 5, the thermal paths toward and away from the body of the patient 32 are illustrated with a series of arrows toward and away from the heating element 12 of the blanket 18. These heat paths can modeled as resistances, as shown in the corresponding model resistance network (FIG. 4), to calculate heat transferred from the heating element 12 though the inner and outer layers of the blanket 18, and to determine the temperature at the middle plane of the blanket 40 (Tᵣₕ). Heat is transferred from the heating element 12 of the blanket 18 through the inner insulator or layer 36 by conduction 52 (R_{ci}=Lᵢᵢ/kᵢA) and then to the body 44 of the patient 32 by radiation 54 (Rᵣᵢ=1/hᵣᵢA) and conduction through the air gap 56 (R_{ca}=L_{ca}/kₐA) through a thin air layer. Heat is also transferred through the outer surface of the blanket 18 by conduction 50 (R_{co}=Lₒᵢ/kᵢA) through the outer insulator or layer 38 and then by natural convection 48 (Rₙₒ=1/hₙₒA) and radiation 46 (Rᵣₒ=1/hᵣₒA) to the environment 42 (Qₑₙᵥ).

Referring again to FIG. 4, the model resistance network for modeling heat transfer in the blanket 18 is shown. To calculate the power to be applied to the blanket to maintain the body temperature at the selected level, losses from outside of the blanket 18 to the environment 42 (Qₑₙᵥ), losses from the head of the patient 32, and hot air leakage from below the blanket are evaluated. Because the losses from a bed below the patient and through a flexible thermoplastic such as polyvinylchloride (PVC) are assumed to be small, these losses can be initially ignored in calculations. Further, heat generated by the wires 16 can be replaced by a uniform heating per unit area since non-uniformities do not play an important role for power considerations. As the air gap Rayleigh number is about 2.5 mm (Ra_{gap}˜1500), convection currents are negligible.

The linear approximation for radiation can be used and the equivalent radiative heat transfer coefficient (hᵣ) assuming large enclosure can be expressed as

hᵣ=4σTₘ³

in which σ is the Stefan-Boltzmann constant, and Tₘ is the average temperature of the two surfaces. This approximation introduces an error below 0.1% in this case and allows modeling the system as a thermal resistance network and solving for temperatures and heat fluxes. Natural convection coefficient for the head (hₙₕ) is estimated with the correlation for the Nusselt number of a sphere

$${Nu}_{D} = \frac{{h}_{nh} {D}_{h}}{{k}_{a}} = 2 + \frac{0.589 {Ra}_{D}^{1 / 4}}{{[ 1 + {( 0.492 / Pr )}^{9 / 16} ]}^{4 / 9}} ;$$

$${Ra}_{D} < {10}^{12}$$

in which Dₕ is the diameter of the head, kₐ is the thermal conductivity of air, Pr is the Prandtl number of air, and Ra_{D} is the Rayleigh number based on the head diameter. The natural convection coefficient for the outer surface of the blanket 18 is estimated with the correlation for a flat plate with heated surface facing up

$$\overline{{Nu}_{W}} = \frac{{h}_{h} W}{{k}_{a}} = 0.14 {Ra}_{W}^{1.3} ;$$

$$2 \times {10}^{7} < {Ra}_{W} < 3 \times {10}^{10}$$

in which W is the width of the heating region, and Ra_{w} is the Rayleigh number based on this width. Conduction resistances are defined in FIG. 5, in which kᵢ is the thermal conductivity of the insulator, Lₒᵢ and Lᵢᵢ are the thicknesses of the inner and outer layers, L_{ca} is an estimated average thickness of the air gap between the blanket and the body, and A is the heating surface of the blanket. Skin resistance is considered negligible compared to the other resistances in this model.

The temperature of the middle plane of the blanket 40 (Tᵣₕ) as a function of the known heat transferred to the body 44 (Q_{b}) needed to keep the head warm is defined as:

$${T}_{rh} = {T}_{b} + {Q}_{b} ( {R}_{ci} + {( \frac{1}{{R}_{ri}} + \frac{1}{{R}_{ca}} )}^{- 1} )$$

From the middle plane temperature, the heat transfer to the environment (Qₑₙᵥ) can be calculated by

$${Q}_{env} = \frac{( {T}_{rh} - {T}_{env} )}{{R}_{co} + {( \frac{1}{{R}_{no}} + \frac{1}{{R}_{ro}} )}^{- 1}}$$

Finally the power requirement to obtain the desired temperature is calculated as the sum of the heat fluxes to the environment and to the body. A correction is added for about 5 L/min hot air leaving the region under the blanket. Power requirement results for a particular embodiment constructed to maintain a temperature of about thirty-seven degrees C. for pediatric patients are discussed below, with reference to table 1.

The minimum time required to reach the desired temperature can be estimated by the time it takes to heat the blanket 18, assuming only one side is exposed to the environment and the other is perfectly insulated. Assuming a lumped capacitance for the blanket, an energy balance per unit area yields

$${ρ}_{i} {c}_{i} {L}_{i} \frac{d T}{d t} = q ( {h}_{ro} + {h}_{no} ) ( T - {T}_{env} )$$

in which ρᵢ and cᵢ are the density and heat capacity of the insulator, and Lᵢ is the total thickness of the blanket (Lₒᵢ+Lᵢᵢ). Assuming the blanket to be initially at environment temperature, this equation can be solved to get the minimum time it takes to reach the desired temperature (T_{d}):

$$t = - \frac{{ρ}_{i} {c}_{i} ( {L}_{oi} + {L}_{ii} )}{{h}_{ro} + {h}_{no}} ln [ 1 - \frac{( {h}_{ro} + {h}_{no} ) ( {T}_{d} - {T}_{env} )}{q} ]$$

As described above, the temperature distribution can be evaluated to assure safe temperatures, particularly adjacent the heating wires 16. To calculate the temperature distribution across the blanket 18, the blanket 18 can be approximated as a fin, except in the region near the wire 16. Heat generation of the wire 16 is replaced by a uniform volumetric heating that has the same width as the thickness of the blanket 18, since isotherms in the wire region are approximately circular. Referring now to FIG. 6, by symmetry, the problem can be reduced to a 1D temperature distribution on a portion of the blanket 17 corresponding to half the spacing between two adjacent wires 16a and 16b (W/2). This section is split into two sections: a first section 60 including the wire 16a with volumetric heating (Qᵥ) and a second section 62 comprising the flexible material of blanket 18, which is a longer distance L without volumetric heating. For the first section, the 1D energy equation yields

$$\frac{{d}^{2} {T}_{1}}{d {x}_{1}^{2}} = \frac{{h}_{o}}{{kL}_{i}} ( {T}_{1} - {T}_{env} ) + \frac{{h}_{i}}{{kL}_{i}} ( {T}_{1} - {T}_{b} ) - \frac{{Q}_{v}}{{k}_{i}}$$

$${h}_{o} = {h}_{ro} + {h}_{no} ; {h}_{i} = {h}_{ri} + \frac{{L}_{ca}}{{k}_{a}}$$

in which x₁ is the distance from the center to the edge of the first section that includes heating, and T₁ is the temperature of the blanket 18 as a function of x₁. This equation can be rewritten using an effective convection coefficient (h_{eff}), and an effective environment temperature (T_{∞}):

$$\frac{{d}^{2} {T}_{1}}{d {x}_{1}^{2}} = \frac{2 {h}_{eff}}{{kL}_{i}} ( {T}_{1} - {T}_{∞} ) - \frac{{Q}_{v}}{{k}_{i}}$$

$${h}_{eff} = \frac{{h}_{o} + {h}_{i}}{2} ; {T}_{∞} = \frac{{h}_{o} {T}_{env} + {h}_{i} {T}_{b}}{{h}_{o} + {h}_{i}}$$

Introducing the fin parameter (m²=2h_{eff}/kᵢLᵢ), the equations for the two sections can be written with their boundary conditions, considering that h_{eff} and T_{∞} are the same for the second section but there is no heat generation:

$$\frac{{d}^{2} {T}_{1}}{d {x}_{1}^{2}} = {m}^{2} ( {T}_{1} - {T}_{∞} ) - \frac{{Q}_{v}}{{k}_{i}}$$

$$\frac{{d}^{2} {T}_{2}}{d {x}_{2}^{2}} = {m}^{2} ( {T}_{1} - {T}_{∞} )$$

$$\frac{d {T}_{1}}{d {x}_{1}} {\vert}_{{x}_{1} = 0} = 0 ; \frac{d {T}_{2}}{d {x}_{2}} {\vert}_{{x}_{2} = L} = 0$$

$$\frac{d {T}_{1}}{d {x}_{1}} {\vert}_{{x}_{1} = i} = \frac{d {T}_{2}}{d {x}_{2}} {\vert}_{{x}_{2} = L} ; {T}_{1} {\vert}_{{x}_{1} = l} = {T}_{2} {\vert}_{x = 0}$$

The first two boundary conditions result from the symmetry conditions and the two others from the continuity of temperature and heat flux at the junction between the two sections. This set of equations is solved to get the temperature distribution. The complete equation is omitted here, but the results were compared to a finite element analysis simulation and showed close correlation. The simulation results show that the approximation of the circular temperature distribution around the wire is adequate and the choice of area for volumetric heat generation is correct.

The maximum increase of temperature due to the wire 16 compared to the average temperature found in the steady state model can be determined from this calculation. With this information it is possible to assure that when the average temperature is at the required level to maintain the selected body temperature, and that the regions directly over wires are not too hot. The maximum temperature difference (ΔTₘ) from the average temperature is given by

$$Δ {T}_{m} = \frac{q}{2 h} [ \frac{I + L}{I} ( 1 - \frac{1}{\frac{sinh ( ml )}{tanh ( ml )} + cosh ( ml )} ) - 1 ]$$

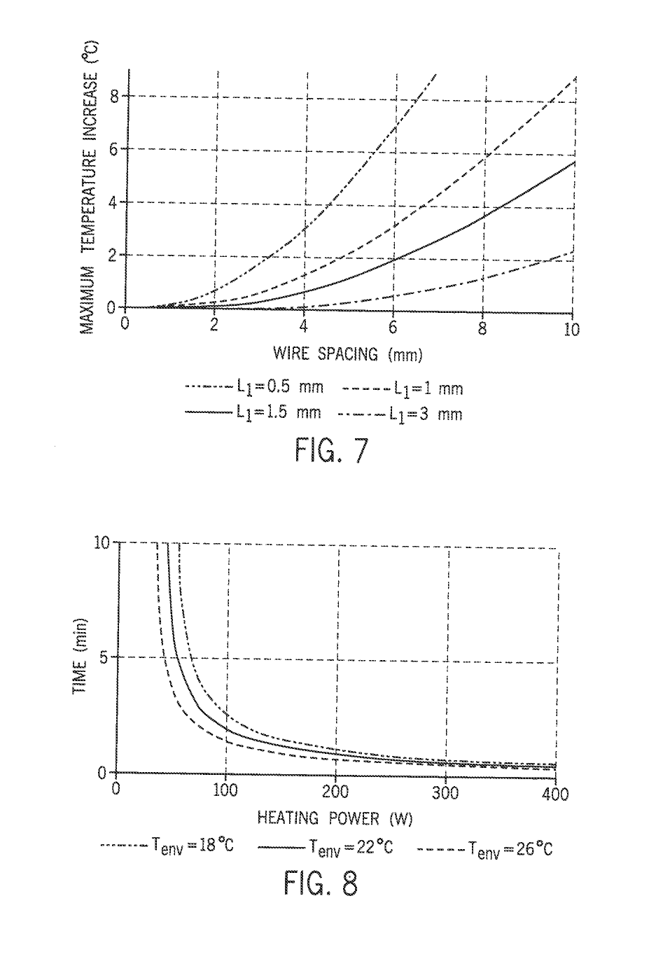

The relation between the maximum increase in temperature compared to average temperature due to the spacing is shown in FIG. 7 for different blanket thicknesses.

As discussed above, the 1D model can be used to assure maximum temperature is sufficiently low to prevent burning of the skin after prolonged exposure. Here, the inner surface is assumed to be in contact with the skin or, at least, near the skin. For some parts of the blanket 18 that are far from the patient 32, the temperature nay exceed this maximum temperature, because the air is insulating the inner surface. The limiting criterion for those areas is that the temperature should decrease to an acceptable level upon contact with the skin. The instantaneous surface temperature (T_{c}) for two solids brought into contact, in this case the flexible material comprising the blanket and skin at temperatures before contact T_{pvc} and Tₛₖᵢₙ, is given by

$$\frac{{T}_{PVC} - {T}_{c}}{{T}_{c} - {T}_{skin}} = \frac{\sqrt{{( k ρ c )}_{skin}}}{\sqrt{{( k ρ c )}_{PVC }}}$$

in which the product (kpc)ₛₖᵢₙ is a measured value for skin, and the product (kpc)_{pvc} is a measured value for the blanket material. A specific example is discussed below.

Using the equations set forth above, warming devices can be constructed for different selected temperatures, designed specifically for infant, child, adult, or other categories of patients or patient needs. In one embodiment, for example, a warming device 10 was advantageously constructed for use to maintain the body of an infant between three and twelve months of age at a temperature of about thirty-seven degrees C., for a duration of about forty-five minutes. Here, to prevent overheating or burning, the blanket 18 is preferably maintained at a temperature below forty-three degrees C. To assure portability, the warming device 10 is designed to be lightweight and transparent, and therefore to enable a medical practitioner to view both the patient 32 and any connections made between the patient and monitoring or other equipment, particularly during transport. Here, the warming device 10 was designed to weigh 4.5 kg or less.

To meet these objectives, the warming device 10 was constructed as a flexible transparent blanket 18 comprising a thermoplastic material, such as polyvinylchloride (PVC). As described above with reference to FIG. 3, the blanket 18 can be constructed of two layers 36 and 38, each 0.75 mm thick. A flexible PVC adhesive can be used to adhere the layers forming the blanket 18 along the peripheral edges. For use with an infant, the blanket 18 was sized to be 900 by 600 millimeters, to provide coverage for an infant up to about twelve months in age.

As described above, the heating element 12 can comprise wires 16 extending between corresponding bus bars 14. Here, the wires 16 were selected to be constructed from nichrome, which is well suited for use in a portable, transparent device. Conductive wires 16 constructed of nichrome are small, lightweight, and provide a high degree of accuracy for resistive heating. The wires 16 are thin, here 0.08 mm, with a resistance rating of 2.5 Ω/cm. The wires 16 are 600 mm in length, providing a series resistance of about 150Ω.

Referring again to FIG. 7, based on the 1.5 mm thickness of the blanket 18, a 6.35 mm spacing was selected for the wires 16, which results in a maximum temperature difference of 2° C. Decreasing the spacing to 4.7 mm would decrease the temperature difference to 1° C., while still offering good visibility. Any further decrease in spacing would lead to small gain, unless the blanket thickness needs to be reduced, in which case a spacing of 2-3 mm would be needed for a 0.5 mm thickness.

To generate an effective resistance of 3Ω such resistances were added in parallel with 6.35 mm spacing. The bus bars 14 are constructed of copper, and 6.35 mm in width. The 1D model, described above, shows that the spacing should not exceed the current 6.35 mm for the thickness of 1.5 mm, to assure the maximum temperature of the inner surface of the blanket in contact with the skin remains within a safe limit of 43° C.

The nichrome wires 16 can be easily bonded to the thermoplastic layers 36 and 38 through melting. PVC, for example, experiences decomposition at 140 degrees C., and begins to melt at around 160 degrees C. By applying the correct amount of power through the wires 16, a localized melting radius develops and provides welding of the wire 16 to the PVC layers 36 and 38. Experimental results indicated that this result could be achieved by applying a power per unit length of 0.44 W/cm for 20 seconds.

The controller 19 was designed to include low voltage battery 20, such as a 12 V DC supply, which can be pulse width modulated to provide power to the heating element 12, as described above. To meet the requirement of portability, the controller 19 was designed to have a battery capacity sufficient for two full 45 min cycles at full power. The controller was also designed to be lightweight (1.4 kg) and to be small enough to fit on the patient's bed (15 cm×10 cm×9 cm). The digital controller 24 can be programmed to provide a timer, which can be set to a pre-determined time frame, such as 45 minutes, or which can be selectively established by the user accessing the controller 24 through, for example, the display 26. Other user input devices such as keyboards and touch pads can also be provided. The heating is preferably limited to 45 minutes, and the capacity of battery 20 can be limited to below 90 minutes.

The digital controller 24 can also be programmed to address electrical risks and risk of hyperthermia. To ensure electrical safety, the resistance of the blanket 18 is verified on startup. A significant change in the resistance of the blanket 18 can indicate broken wires 16. For example, the controller 24 may be programmed to take corrective action when the resistance increased by 50% or more. The digital controller 24 can also be programmed to address the risk of hyperthermia, which can arise if the power controller 19 is disconnected from the heating element 12 of the blanket 18, or if the voltage of battery 20 drops. The controller 24 can either issue an alert to the user, turn off the power to the heating element 12 of the blanket 18, or both if the resistance of the blanket increases substantially

Power requirements for this embodiment, as determined using the equations described above are shown in table 1 below, where the thermal conductivity of the insulator for the selected material is 0.16 W/m²K. Assuming an ambient temperature of about twenty two degrees C., the power controller 19 needs to deliver about 50 W of power.

| Ambient | Inside blanket | Outside surface | Total power |
|---|---|---|---|
| temperature | temperature | temperature | requirement |
| (° C.) | (° C.) | (° C.) | (W) |
| 14 | 40.9 | 42.1 | 81 |
| 18 | 40.2 | 41.1 | 65 |
| 22 | 39.5 | 40.2 | 50 |
| 26 | 38.8 | 39.3 | 35 |

Referring now to FIG. 8, a chart illustrating the minimum time required to heat a blanket 18 constructed as described above from environmental temperature to forty degrees C. is shown. For the materials used here, ρᵢ and cᵢ, the density and heat capacity of the insulator, are 1714 kg/m³ and 1050 J/kgK, respectively. As can be seen in the chart, heating the blanket 18 quickly, and particularly over a period of a minute or less, requires significantly higher power than maintaining the steady state temperature. About 4 times the steady state power would be needed to heat the blanket to heat the blanket from room temperature to the desired temperature of about 40° C. in a time below 2 mm. To achieve a fast heating cycle, therefore, additional batteries or power may be desirable for a turn-on period. For example, an external power source, such as an inverter power supply powered through a stationary power supply, or a direct connection to a power supply from a wall socket, could be used to initially or indefinitely power the blanket 18. When the temperature reaches an adequate temperature for steady-state performance, the power supply can be switched to the battery 20, particularly for transporting the patient.

When a warming device 10 as described above was operated in steady state tests at different power levels including 12 V, 16 V, 24 V, and 31 V, the time to reach a temperature of 40° C. took respectively 14 min, 5 min, 2.5 min and 2 min. Indeed it was found during the steady state testing that the device should operate close to 12 V to deliver the required power which would mean a pre-heating time of 14 min. A heating cycle of 2.5 min at 200 W (24 V) will reach the desired steady-state temperature more quickly.

The warming device described above can be easily constructed using the following steps: (1) cut two layers 36 and 38 of PVC to the selected size, (2) place the first layer 36 on a tooling board providing appropriate spacing for the bus bars and wires, (3) position copper bus bars 14 on the layer 36, (4) wrap nichrome wires 16 to create the resistive heating element 12, (5) position the second layer 38 of PVC to form the blanket 18, (6) connect the power controller 19 to the heating element 12, and (6) apply adhesive to the outer edges. In one specific embodiment, tooling can be fabricated using CNC, and ¼″ spring pins can be applied to a plywood board to align the nichrome wires 16.

The disclosure therefore describes a transparent portable warmer device, which can be advantageously applied to insure patient normothermia during medical transport, which can be advantageously applied to infants and other pediatric applications. The device can be constructed as a blanket 18 made from two thin layers 36, 38 of flexible transparent PVC bonded together by heating nichrome wires 16 placed between the layer. A power controller 19 can be applied to keep the patient warm, and can include batteries 20 capable of providing the desired power during a 90 min period.

A minimum heating power of 50 W was determined by heat transfer analysis to provide steady state operation to maintain a target temperature of thirty seven degrees C. A 1D model for the blanket 18 was used to determine that the spacing between the wires should be about 6.35 mm for a selected thickness of 1.5 mm, and that this selected thickness and spacing would establish a maximum temperature at the inner surface of the blanket 18 in contact with the skin to be about 43° C.

For this embodiment, the instantaneous surface temperature T_{c} can be calculated using the equation above, where (kpc)ₛₖᵢₙ is measured to be 1.3×106 J/m⁴sK and (kpc)_{pvc} is 2.8×105 J/m4sK. Far from the skin, assuming the inner surface to be perfectly insulated, the maximum temperature of the blanket can reach 47° C., for the heat flux needed in steady state. Assuming the infant skin temperature to be 37° C., the instantaneous contact temperature is 40° C., which is safe for an infant.

Experiments show the great transparency of the device and the adequate flexibility, and confirm the heat transfer analysis results. Steady state power, tested with an aluminum U-shape extrusion, is in good agreement with the model. Time to heat the blanket exceeds the predicted one, but shows the same trend. Temperature distribution measured with a thermal camera showed a difference below 0.5° C. from the 1D model. Preliminary testing shows that the temperature of the blanket drops to an acceptable level when brought in contact with the skin, even if the temperature before contact exceed 43° C. The system was operated experimentally during 70 min period using a 1.5 kg battery, satisfying the portability requirement.

While present inventive concepts have been described with reference to particular embodiments, those of ordinary skill in the art will appreciate that various substitutions and/or other alterations may be made to the embodiments without departing from the spirit of present inventive concepts. For example, although a PVC material is described, silicone rubber, vinyl, and PET (Polyethylene Terephtalate) flexible materials can also be used. Further, although nichrome wires are described above, various types of metal and other resistive current-carrying materials can be applied as part of the heating element. Accordingly, the description herein is meant to be exemplary, and does not limit the scope of present inventive concepts. A number of examples have been described herein. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the present inventive concepts.

I claim:

1. A portable transparent warming device comprising:
a flexible transparent layer sized and dimensioned to cover a portion of a body of a medical patient;
a plurality of resistive wires integrated with the flexible transparent layer; and
a power controller electrically connected to the resistive wires so to apply a current to sufficient to produce heat from the resistive wires, wherein heat produced from the wires is sufficient to achieve and maintain a steady state temperature of about 40 degrees Celsius beneath a substantial portion of the flexible transparent layer.

2. The portable transparent warming device of claim 1, wherein the power controller is sized to produce a steady state temperature of about 40 degrees within an environment having an ambient temperature of about 23 degrees Celsius or less.

3. The portable transparent warming device of claim 1, wherein the power controller is sized to provide sufficient power to the resistive heating element to raise the temperature of the flexible transparent layer from ambient to a predetermined steady state temperature within a time period of about 14 minutes.

4. The portable transparent warming device of claim 1, wherein the steady state temperature can be achieved within a time period of about 2 minutes.

5. The portable transparent warming device of claim 1, wherein the power controller comprises a battery providing a power output of about 12 Volts.

6. The portable transparent warming device of claim 1, wherein the power controller comprises a battery providing a power output of about 24 Volts.

7. The portable transparent war device of claim 1, wherein the plurality of resistive wires comprises nichrome.

8. The portable transparent warming device of claim 1, wherein the at least one flexible transparent layer comprises transparent polyvinylchloride (PVC).

9. The portable transparent warming device of claim 1, wherein the power controller further includes a digital controller programmed to regulate the power output of the resistive wires.

10. The portable transparent warming device of claim 9, wherein the power controller comprises a switch electrically connected to the resistive wires and the digital controller, the digital controller configured to activate and deactivate the switch to produce a pulse-width modulated power supply for controlling the heat of the resistive wires.

11. The portable transparent warming device of claim of claim 9, wherein the digital controller limits maximum power output to limit a surface temperature of a portion of the blanket directly adjacent skin to about 38 degrees Celsius or less.

12. The portable transparent warming device of claim 9, wherein the switch comprises a MOSFET.

13. The portable transparent warming device of claim 1, wherein the at least one flexible transparent layer comprises two transparent layers and the resistive wires are arranged in a parallel circuit configuration between the layers.

14. The portable transparent warming device of claim 1, wherein the warming device weighs 4.5 kg or less.

15. The portable transparent warming device of claim 1, wherein In an embodiment, the battery weighs about 1.5 Kg or less.

16. The portable transparent warming device of claim 1, wherein the at least one transparent layer is sufficiently sized to cover at least an infant.

17. The portable transparent warming device of claim 16, wherein the at least one transparent layer is about 600 by 900 millimeters in size.

18. The portable transparent warming device of claim 1, wherein the warming blanket can be powered from a dedicated constant power source.

19. The portable transparent warming device of claim 1, wherein the heating element is coupled to a power source from a wall socket to initially warm the blanket, then unplugged and switched to battery power while the blanket and person thereunder is transported or moved.

20. The portable transparent warming device of claim 1, wherein the resistive wires are connected in a parallel circuit between copper bus bars.
